(19) United States
(12) Patent Application Publication
(10) Pub. No.: US 2025/0064618 A1
(43) Pub. Date: Feb. 27, 2025

(54) WEARABLE STABILIZATION ASSEMBLY AND SYSTEM

(71) Applicant: GM Global Technology Operations LLC, Detroit, MI (US)

(72) Inventors: Elise Renee Friedman, Royal Oak, MI (US); Edeena Alicia Reader, Canton, MI (US)

(73) Assignee: GM Global Technology Operations LLC, Detroit, MI (US)

(21) Appl. No.: 18/456,260

(22) Filed: Aug. 25, 2023

(51) Int. Cl. A61F 5/01; A61B 5/00; A61B 5/11

(52) U.S. Cl. CPC A61F 5/0118; A61B 5/1125; A61B 5/6826; A61F 2005/0169

(57) ABSTRACT

A wearable stabilization assembly includes a sensor and a tensioning assembly that is operably coupled to the sensor. A mass is operably coupled to the tensioning assembly. The stabilization assembly also includes a power unit that is electrically coupled to the tensioning assembly, and a controller is communicatively coupled with the sensor. The controller is configured to activate the power unit to oscillate the mass via the tensioning assembly when a variable detected by the sensor is outside a predetermined variable parameter.

### INTRODUCTION

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against present disclosure.

The present disclosure relates generally to a stabilization assembly.

The human body includes thousands of neurological pathways that are utilized in executing bodily movements. These neurological pathways, while often associated with intentional bodily movement, may result in unintentional bodily movement. For example, tremors, dyskinesias, or shaking of appendages or the entire body may occur, which may be perceived as vibrations. While an individual may attempt to control tremors, often the tremors are a result of neurological conditions that may restrict the individual's ability to stop or otherwise control the tremors.

In some conditions, unintended bodily movement may be a persistent condition that the individual attempts to manage on a continual basis. In other conditions, the tremors may be intermittent in that the individual may not expect a tremor until the onset of the tremor. A rapid onset of tremors may impact day-to-day activities of the individual. For example, unintentional bodily movements, such as tremors, may present challenges in operating vehicles or other equipment.

### SUMMARY

In some configurations, a wearable stabilization assembly includes a housing that includes a first sensor disposed at a first end of the housing. A tensioning assembly is disposed within the housing and is operably coupled to the first sensor via a connection extending through the tensioning assembly. The tensioning assembly includes a first tensioning element, a second tensioning element, and a tensioning device that is operably coupled to each of the first tensioning element and the second tensioning element. The wearable stabilization assembly also includes a mass that is operably coupled to each of the first tensioning element and the second tensioning element. A controller is communicatively coupled with the first sensor and, in response to the first sensor detecting a variable that is outside a predetermined variable parameter, is configured to translate the mass via the tensioning assembly.

In some aspects, the housing may include a first retention feature that may include the first sensor and a second retention feature that may include a second sensor. The controller may be configured to receive a first vibration variable from the first sensor and a second vibration variable from the second sensor an may be configured to compare the first vibration variable with the second vibration variable. The controller may also be configured to activate the tensioning device to bias the first tensioning element and the second tensioning element when both the first variable and the second variable outside the predetermined variable parameter. In some examples, the wearable stabilization assembly may include a power unit that is electrically coupled to each of the first sensor, the controller, and the tensioning device. The controller, in response to the predetermined variable parameter detected by the first sensor, may be configured to activate the power unit and bias the first tensioning element and the second tensioning element via electrical communication between the power unit and the tensioning device. In further examples, the second tensioning element may be disposed on an opposite side of the mass than the first tensioning element. Each of the first tensioning element, the mass, and the second tensioning element may be operably coupled in series.

In another configuration, a stabilization assembly for an appendage includes a sensor and a tensioning assembly that is operably coupled to the sensor. A mass is operably coupled to the tensioning assembly. The stabilization assembly also includes a power unit that is electrically coupled to the tensioning assembly, and a controller is communicatively coupled with the sensor. The controller is configured to activate the power unit to oscillate the mass via the tensioning assembly when a variable detected by the sensor is outside a predetermined variable parameter.

In some examples, each of the sensor, the tensioning assembly, the mass, the power unit, and the controller may be operably coupled in series. In some aspects, the tensioning assembly may include a tensioning device and a first tensioning element operably coupled to the tensioning device. The controller may be configured to bias the first tensioning element in response to the detected variable being outside the predetermined variable parameter. The controller may also be configured to activate the tensioning device to dampen the detected variable to a variable that may be within the predetermined variable parameter. The tensioning assembly may include a second tensioning element and the controller may be configured to activate the tensioning device to oscillate the mass between the first tensioning element and the second tensioning element to dampen the detected variable. Optionally, the first tensioning element and the second tensioning element may be arranged in parallel with the mass. In other aspects, the mass may be a fluid-filled mass.

In another configuration, a wearable stabilization system includes a first stabilization device including a first sensor, a first tensioning assembly, a first mass, and a first controller that is communicatively coupled to the first sensor. The first controller is configured to oscillate the first mass via the first tensioning assembly in response to a variable detected by the first sensor being outside a predetermined variable parameter. The wearable stabilization system also includes a second stabilization device that includes a second sensor, a second tensioning assembly, a second mass, and a second controller that is communicatively coupled to the second sensor and the first controller. The second controller is configured to oscillate the second mass via the second tensioning assembly in response to at least one of the first controller oscillating the first mass and a variable detected by the second sensor being outside the predetermined variable parameter.

In some aspects, the second controller may be communicatively coupled to the first controller. The second controller may be configured to receive a signal from the first controller that may correspond to the variable detected by the first sensor. The second controller may also be configured to activate a power unit of the second stabilization device to bias the second tensioning assembly. Each of the first controller and the second controller may be configured to reduce the detected variables by activating the first tensioning assembly and the second tensioning assembly, respectively. In some examples, the second controller may be configured to activate the second tensioning assembly in response to the signal received from the first controller.

The wearable stabilization system may further include a third stabilization device that may include a fluid-filled mass, an adjustment valve, and a third controller. The third controller may be communicatively coupled with each of the first controller and the second controller. The third controller may be configured to adjust a volume of fluid within the fluid-filled mass via the adjustment valve in response to a signal received from at least one of the first controller and the second controller. In other aspects, a vehicle may include an electronic control unit that may be communicatively coupled with the wearable stabilization assembly.

### BRIEF DESCRIPTION OF THE DRAWINGS

The drawings described herein are for illustrative purposes only of selected configurations and are not intended to limit the scope of the present disclosure.

FIG. 1 is an enlarged partial elevation view of an example stabilization assembly according to the present disclosure;

FIG. 2 is a perspective view of an individual wearing a stabilization assembly according to the present disclosure in communication with a third party device;

FIG. 3A is an enlarged partial perspective view of a hand of the individual in FIG. 1 with a stabilization assembly according to the present disclosure;

FIG. 3B is an enlarged partial perspective view of a hand with a stabilization assembly according to the present disclosure;

FIG. 4 is a side elevation view of a stabilization assembly according to the present disclosure with a housing;

FIG. 5A is a side elevation view of a stabilization assembly according to the present disclosure free from a housing;

FIG. 5B is a perspective view of the stabilization assembly of FIG. 5A;

FIG. 6 is a perspective view of a tensioning element of a stabilization assembly according to the present disclosure;

FIG. 7 is a perspective view of a mass of a stabilization assembly according to the present disclosure;

FIG. 8 is a functional block diagram of an example stabilization assembly providing communication between a controller, a sensor, and a tensioning assembly according to the present disclosure; and

FIG. 9 is an example method of executing a stabilization protocol according to the present disclosure.

Corresponding reference numerals indicate corresponding parts throughout the drawings.

### DETAILED DESCRIPTION

Example configurations will now be described more fully with reference to the accompanying drawings. Example configurations are provided so that this disclosure will be thorough, and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

In this application, including the definitions below, the term module may be replaced with the term circuit. The term module may refer to, be part of, or include an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor (shared, dedicated, or group) that executes code; memory (shared, dedicated, or group) that stores code executed by a processor; other suitable hardware components that provide the described functionality; or a combination of some or all of the above, such as in a system-on-chip.

The term code, as used above, may include software, firmware, and/or microcode, and may refer to programs, routines, functions, classes, and/or objects. The term shared processor encompasses a single processor that executes some or all code from multiple modules. The term group processor encompasses a processor that, in combination with additional processors, executes some or all code from one or more modules. The term shared memory encompasses a single memory that stores some or all code from multiple modules. The term group memory encompasses a memory that, in combination with additional memories, stores some or all code from one or more modules. The term memory may be a subset of the term computer-readable medium. The term computer-readable medium does not encompass transitory electrical and electromagnetic signals propagating through a medium, and may therefore be considered tangible and non-transitory memory. Non-limiting examples of a non-transitory memory include a tangible computer readable medium including a nonvolatile memory, magnetic storage, and optical storage.

The apparatuses and methods described in this application may be partially or fully implemented by one or more computer programs executed by one or more processors. The computer programs include processor-executable instructions that are stored on at least one non- transitory tangible computer readable medium. The computer programs may also include and/or rely on stored data.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

The non-transitory memory may be physical devices used to store programs (e.g., sequences of instructions) or data (e.g., program state information) on a temporary or permanent basis for use by a computing device. The non-transitory memory may be volatile and/or non- volatile addressable semiconductor memory. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

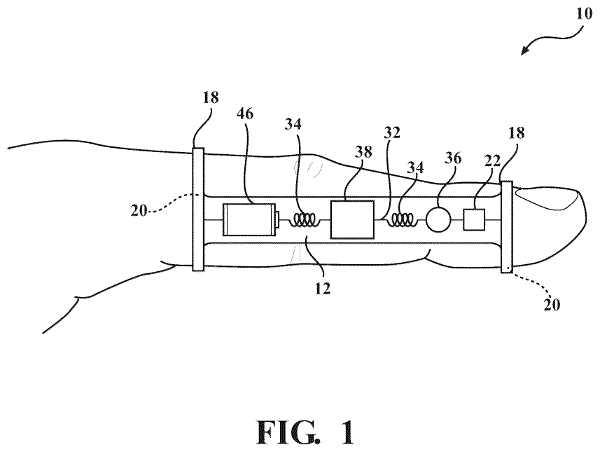

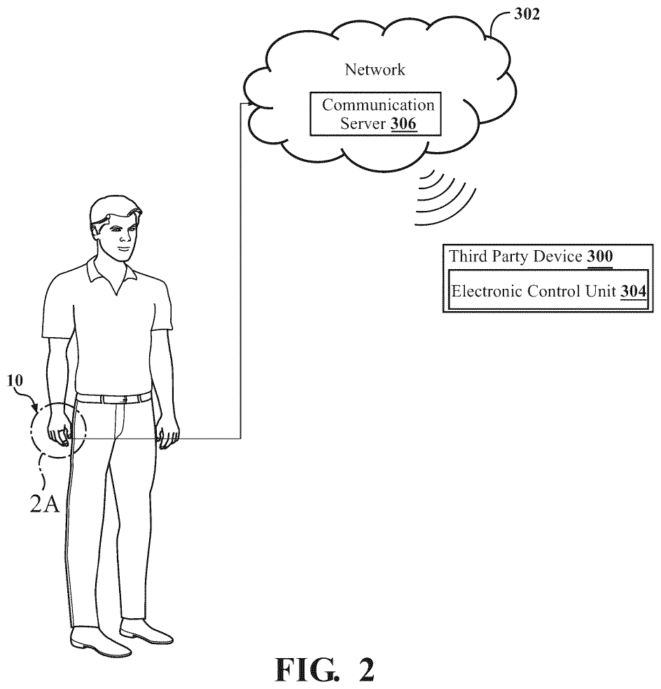

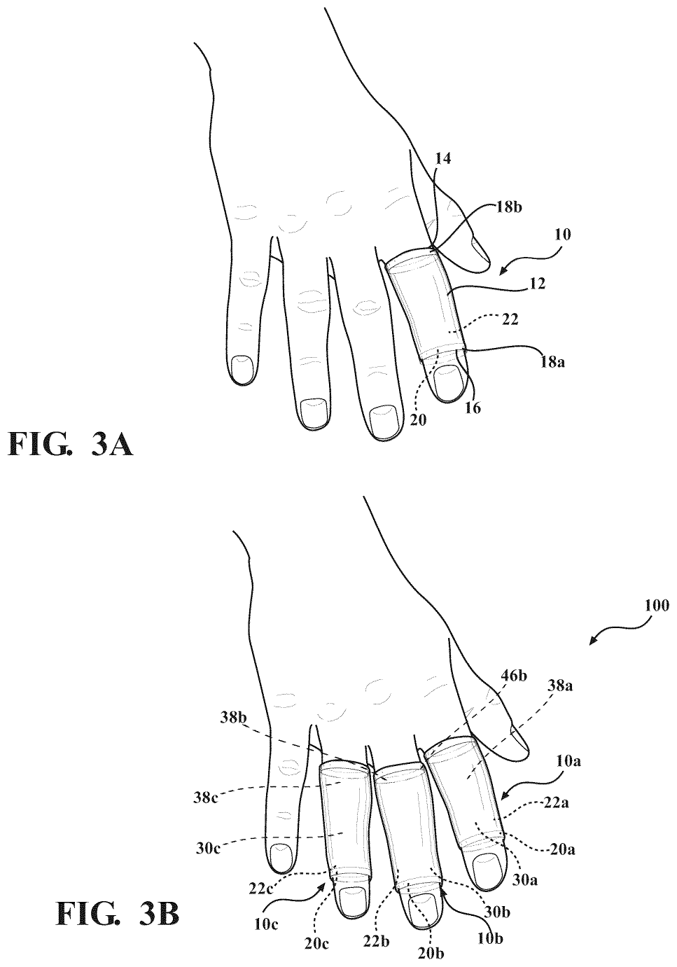

Referring to FIGS. 1-5B, a stabilization device or assembly 10 is illustrated as being positioned on a body of a wearer. The stabilization assembly 10 is illustrated throughout as being positioned or otherwise attached to one or more digits of the wearer. However, it is contemplated that the stabilization assembly 10 may be used in any practicable location on the body of the wearer. For example, in some aspects, the stabilization assembly 10 may be positioned along an arm, a leg, or a torso of the wearer. In some examples, the stabilization assembly 10 may be used with alternate organic and/or inorganic bodies including, but not limited to, a tree and a prosthetic appendage. Accordingly, the size of the stabilization assembly 10 may be adjusted to the corresponding appendage or body location of the wearer or adjusted to the corresponding inorganic body. It is also contemplated that more than one stabilization assembly 10 may be utilized and worn by the wearer. As illustrated in FIG. 3B, the wearer may have a stabilization assembly 10 on each phalange. As described in more detail below, the combination of multiple stabilization assemblies 10a-10c may be defined as a stabilization system 100. The stabilization system 100 may include one or more stabilization assemblies 10a-10c that cooperate to dampen a detected variable 200, as described herein.

While the stabilization assemblies 10a-10c are illustrated on digits of the wearer, each stabilization assembly 10a-10c may be positioned in various locations, as noted above. By way of example, not limitation, a first stabilization assembly 10a may be positioned on a digit and a second stabilization assembly 10b may be positioned on an arm. The position of the respective stabilization assembly 10a-10c may correspond to the degree of dampening anticipated by the stabilization system 100. In further examples, each stabilization assembly 10a-10c of the stabilization system 100 may be communicatively coupled and configured to cooperate to maximize the effects of each stabilization assembly 10, described in more detail below.

The stabilization assembly 10 may include a housing 12 having a first end 14 and an opposing second end 16. Each of the first end 14 and the second end 16 may be equipped with a retention feature 18 configured to retain the stabilization assembly 10 on the wearer. In some configurations, the stabilization assembly 10 may be free from a housing 12, such that each element of the stabilization assembly 10 may be suspended between the retention features 18 and generally exposed to the surrounding environment. Additionally or alternatively, the housing 12 may be configured to encase the elements of the stabilization assembly 10 described herein.

With reference to FIGS. 3-5B, the retention features 18 of the stabilization assembly 10 may include at least one sensor 20 disposed within one or more of the retention features 18. For example, a first retention feature 18a may include a first sensor 20a and a second retention feature 18b may include a second sensor 20b. In other aspects, the stabilization assembly 10 may include a single sensor 20 disposed in one of the retention features 18. In further aspects, the sensor 20 may be separate from the retention features 18 and the housing 12. It is contemplated that the sensors 20 may be configured as mechanical sensors, electrical sensors, and/or any combination of mechanical sensors and electrical sensors. In a non-limiting example, the sensors 20 may be electrodes positioned along a head of the wearer and are configured to measure electrical activity of the brain in the form of an electroencephalogram (EEG). In this configuration, the sensors 20 are in wireless communication with a controller 22 to communicate the detected electrical inputs 200 that correspond to the brain measurements measured by the EEG. In an additional configuration, the controller 22 and/or the one or more sensor(s) 20 may be separate from the retention features 18. For example, the sensor 20 may be positioned along a neural pathway of the wearer. Additionally or alternatively, the sensors 20 may be mounted on any practicable location along the body.

In other examples, the sensors 20 may be one or more mechanical sensors, which may include an accelerometer and a gyroscope, which detects variables 200. In either configuration of the sensors 20, the sensors 20 are configured to detect a variable 200, and the stabilization assembly 10 utilizes the detected variable 200 to selectively dampen the bodily movement. The variable 200 may include, but is not limited to, a frequency, an amplitude, an acceleration, and an angle. In some examples, a first variable 200a may be detected by the first sensor 20a and a second variable 200b may be detected by the second sensor 20b. Each sensor is configured to detect one or more variables 200. The second sensor 20b may be utilized as a confirmation to identify whether the variable 200 detected has been dampened by the stabilization assembly 10.

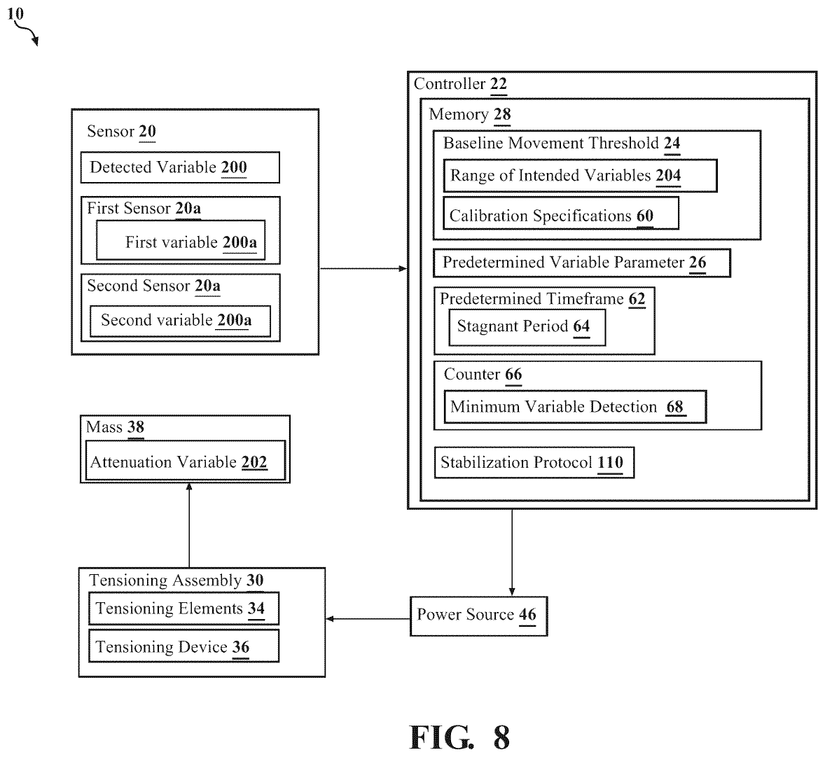

As described above, the controller 22 is configured to receive the detected variable 200 from the sensor 20. In some examples, the controller 22 may be configured to receive the first vibration variable 200a from the first sensor 20a and the second vibration variable 200b from the second sensor 20b and may then compare the first vibration variable 200a with the second vibration variable 200b. The controller 22, outlined in FIG. 8, stores a baseline movement threshold 24 and a predetermined variable parameter 26 on a memory 28 of the controller 22. The controller 22 is also configured with a stabilization protocol 110 that may be stored on the memory 28 and utilizes each of the baseline movement threshold 24 and the predetermined variable parameter 26 in comparison with the detected variable 200, as described in more detail below.

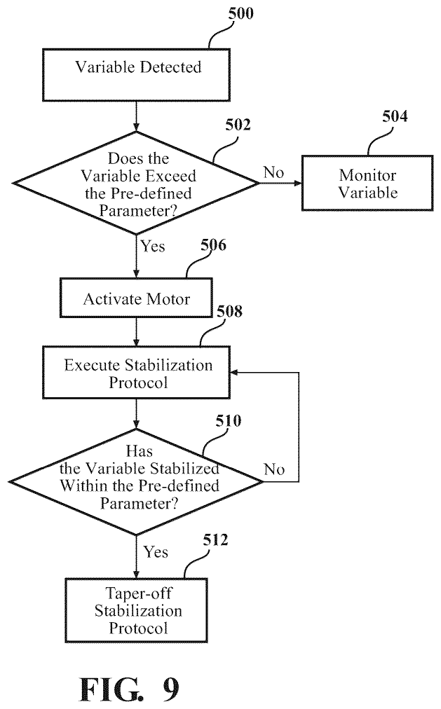

For example, the controller 22 may compare the detected variable 200 received from the sensor 20 with one or both of the baseline movement threshold 24 and the predetermined variable parameter 26 to determine whether to activate the stabilization protocol 110. If the detected variable 200 is outside the predetermined variable parameter 26, then the controller 22 will activate the stabilization protocol 110. The stabilization protocol 110 is described in more detail with reference to FIGS. 7-9 in combination with the baseline movement threshold 24 and the predetermined variable parameter 26.

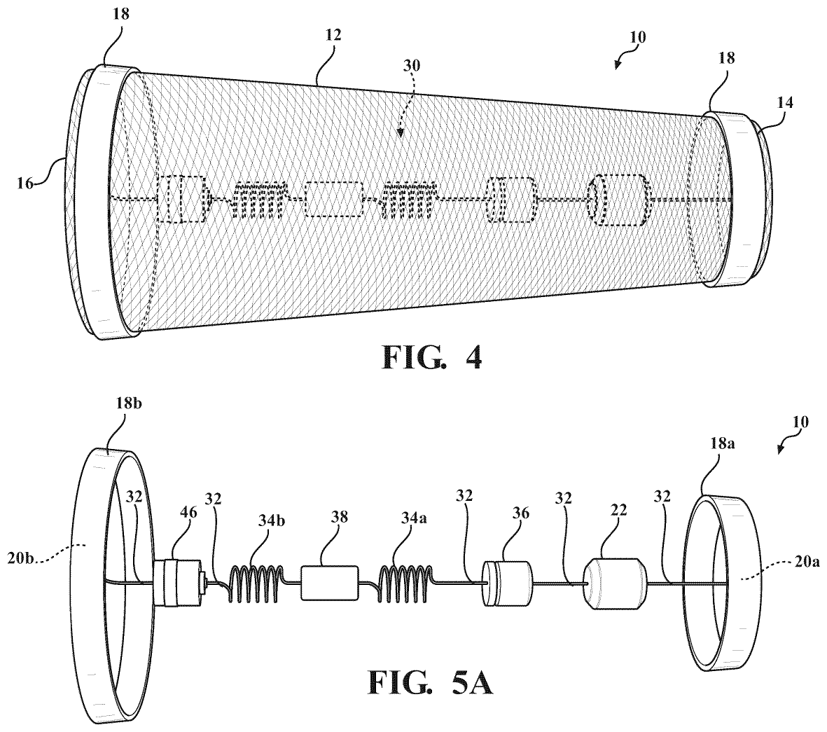

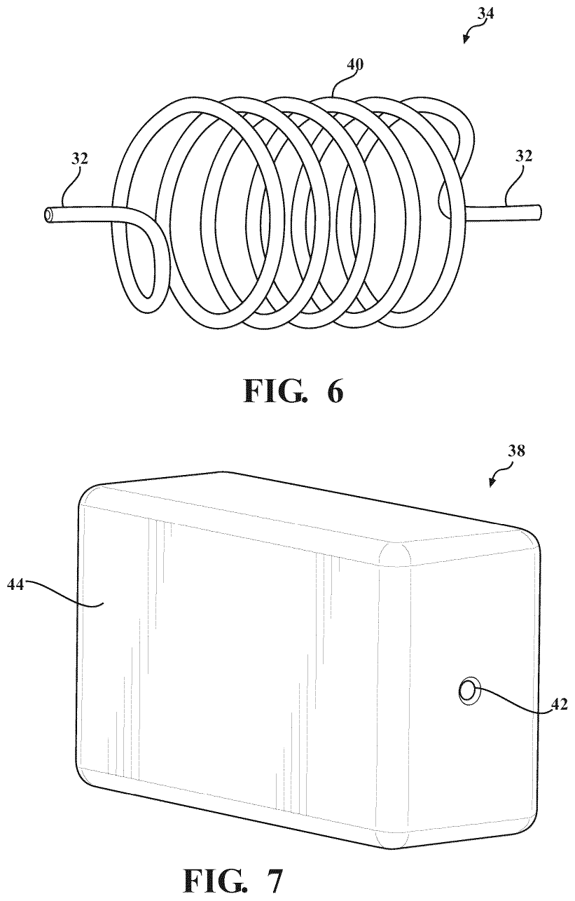

With reference to FIGS. 4-7, the stabilization assembly 10 also includes a tensioning assembly 30 that is disposed within the housing 12 and is operably coupled to the controller 22 and the sensor 20 via a connection 32 extending through the tensioning assembly 30. The tensioning assembly 30 includes tensioning elements 34 that may include a first tensioning element 34a and a second tensioning element 34b. Additionally or alternatively, more than two tensioning elements 34 or a single tensioning element 34 may be utilized with the stabilization assembly 10. A tensioning device 36 is operably coupled to the tensioning element 34 and is configured to bias the tensioning elements 34 when the detected variable 200 is outside the predetermined variable parameter 26. For example, the tensioning device 36 may be a motor that biases the tensioning elements 34 in response to the controller 22 executing the stabilization protocol 110. While described as a motor, the tensioning device 36 may also be configured as shaped metal alloy elements, electrorheological elements, or the like. The tensioning elements 34 are illustrated as having a spring configuration and are positioned on either side of a mass 38. In some examples, the tensioning elements 34 have coils 40 and may be integrally formed with the connection 32. The tensioning elements 34 may include, but are not limited to, springs, cables, hyperelastic shaped metal alloys, shaped metal alloys, and any other practicable tensioning elements.

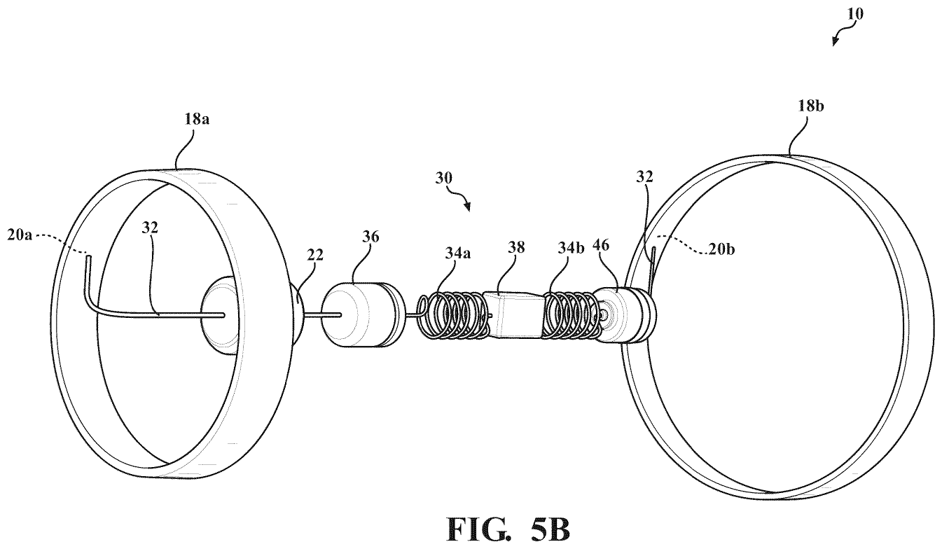

The tensioning elements 34 may have other configurations, such that the tensioning elements 34 are not limited to a spring configuration. The tensioning elements 34 are configured to bias or oscillate the mass 38. For example, the power unit 46 provides a current to the tensioning device 36 to compress and expand each of the tensioning elements 34 positioned on either side of the mass 38. The tensioning device 36 may, for example, be configured as a motor that may provide variable tensioning to induce and release stiffness to the tensioning elements 34. The resultant current along the tensioning elements 34 may bias the mass 38 to neutralize the detected variable 200. As depicted in FIGS. 5A and 5B, the tensioning elements 34 and the mass 38 are arranged in series, such that the first tensioning element 34a is positioned on an opposing side of the mass 38 from the second tensioning element 34b. For example, the mass 38 may be suspended between the first tensioning element 34a and the second tensioning element 34b with the connection 32 extending through an aperture 42 of the mass 38. With the connection 32 extending through the aperture 42, the mass 38 may travel along a single axis between the tensioning elements 34.

It is also contemplated that, in some examples, the tensioning elements 34 and the mass 38 may be arranged in parallel, such that more than two tensioning elements 34 may be utilized to surround the mass 38. In the parallel configuration, the mass 38 may be biased by the tensioning elements 34 and may travel along multiple axes to dampen or otherwise attenuate the detected variable 200. In some examples, six tensioning elements 34 may be utilized to bias the mass 38. The mass 38 may be a solid component with a predefined weight to counterbalance and otherwise attenuate the detected variable 200 at the attenuation variable 202. In some examples, the mass 38 may be a fluid-filled mass 38. For example, the mass 38 may include an outer casing 44 in which a fluid may be disposed. In this example, the apertures 42 may include one-way valves through which the connection 32 may pass while the fluid is retained within the outer casing 44. Optionally, the fluid may be adjusted using a pump (not shown) to reach a selected fluid level that may facilitate attenuation of the detected variable 200.

Referring still to FIGS. 4-7, the connection 32 may be defined with a predefined tension that is configured to oscillate the mass 38 at a selected attenuating variable 202. In some examples, the tension of the connection 32 may be adjusted via the tensioning assembly 30, in that the tensioning elements 34 may be tightened or loosened by the tensioning device 36 depending on the selected attenuation variable 202. Vibrations along the stabilization assembly 10 are continuously sampled by the sensor 20 and monitored by the controller 22 to activate the tensioning assembly 30 as needed.

The stabilization assembly 10 also includes a power unit 46 that is electrically coupled to the controller 22 and the tensioning device 36. The power unit 46 may also be electrically coupled to the sensor 20. The power unit 46 is depicted in series along the connection 32 with each of the tensioning elements 34, the mass 38, the tensioning device 36, and the controller 22. In some aspects, the power unit 46 may be coupled to but separate from the stabilization assembly 10. The power unit 46 may provide electrical power to the tensioning device 36 in response to activation by the controller 22 to activate the tensioning device 36 and ultimately translate the tensioning elements 34 between a compressed state and an extended state. For example, the controller 22 may activate the power unit 46 when the stabilization protocol 110 is executed to bias the first tensioning element 34a and the second tensioning element 34b via electrical communication between the power unit 46 and the tensioning device 36. The power unit 46 may be configured as any practicable device configured to transmit power to the tensioning device 36.

Referring to FIGS. 7-9, the stabilization assembly 10 is a wearable stabilization assembly 10 and is configured to detect variables proximate to the location of the stabilization assembly 10 on a wearer. As noted above, the sensor 20 may be configured to detect electrical inputs or may be configured to detect mechanical inputs. The illustrated example provides the example of the sensor 20 detecting vibration frequencies. The same or similar description may be attributed to an electrical variable with the primary difference being the location of the sensor 20. For example, in either configuration, the sensor 20 detects the variable 200 and transmits the detected variable 200 to the controller 22.

Vibrations are transmitted through various movements of the wearer including, but not limited to, tapping of fingers, waving of hands or arms, walking, tapping of feet, and/or any other bodily movement. Each vibration has a variable 200 that is detected by the sensor 20. Intended movements typically have a variable 200 that is constant or otherwise within the predetermined variable parameter 26. For example, most intended movements may be categorized at or approximate to the baseline movement threshold 24. The baseline movement threshold 24 may be utilized in calibrating the predetermined variable parameter 26, with both the baseline movement threshold 24 and the predetermined variable parameter 26 stored in the memory 28 of the controller 22.

The baseline movement threshold 24 may serve as a marker for the controller 22 when activating the stabilization protocol 110. The stabilization protocol 110 is configured to activate the tensioning assembly 30 to oscillate the mass 38 and attenuate the detected variable 200. For example, the sensor 20 may detect a variable 200 that is outside the predetermined variable parameter 26 and, in response, the controller 22 may activate the tensioning assembly 30 until the detected variable 200 is within the predetermined variable parameter 26. That is, the stabilization protocol 110, via the stabilization assembly 10, is configured to stabilize movement of the wearer without preventing intended movements.

The baseline movement threshold 24 may be configured with a range of intended variables 204 and calibration specifications 60. The range of intended variables 204 may be utilized by the controller 22 when evaluating the detected variables 200. As mentioned above, the placement of the sensor 20 may determine the variable 200 detected, such that some placements may have a naturally higher movement variable as compared to other placements. Thus, the range of intended variables 204 may be utilized to provide various reference points in determining the baseline movement threshold 24.

It should be noted that each of the baseline movement threshold 24, the range of intended variables 204, and the predetermined variable parameter 26 may be specific to the wearer. Accordingly, the baseline movement threshold 24 includes the calibration specifications 60, such that the stabilization assembly 10 may be specifically calibrated for the wearer. The calibration specifications 60 may also be utilized to calibrate to a particular location of the stabilization assembly 10. For example, the stabilization assembly 10 may be positioned on a digit of the wearer, which may have different calibration specifications 60 as compared to the stabilization assembly 10 being positioned on a foot of the wearer.

With further reference to FIGS. 7-9, the controller 22 may be configured with a predetermined timeframe 62, which may be defined as a period of time during which the detected variable 200 is outside the predetermined variable parameter 26. The predetermined timeframe 62 assists the controller 22 in determining, when the detected variable 200 is outside the predetermined variable parameter 26, whether the detected variable 200 was an isolated incident or a repeated, unintended movement. While the predetermined timeframe 62 is a sufficient duration of time in which the controller 22 may identify the detected variable 200 as a repeated, unintended movement, the predetermined timeframe 62 is a short enough period of time in which the controller 22 may execute the stabilization protocol 110 with minimal delay. The predetermined timeframe 62 may also be configured with a stagnant period 64 during which the detected variable 200 is not outside the predetermined variable parameter 26. The stagnant period 64 is defined as a period of time, predetermined and stored in the memory 28 of the controller 22, when each detected variable 200 is within the predetermined variable parameter 26. The stagnant period 64 may be reset after each detected variable 200 that is outside the predetermined variable parameter 26.

The controller 22 may also include a counter 66 to determine how often the detected variable 200 is outside the predetermined variable parameter 26 within the predetermined timeframe 62. In some examples, the counter 66 may be configured with a minimum variable detection 68, which corresponds to a preset number of times that the detected variable 200 is outside the predetermined variable parameter 26 within the predetermined timeframe 62. Thus, the controller 22 may, based on the counter 66 and the predetermined timeframe 62, wait to activate the stabilization protocol 110 until the detected variables 200 exceed the minimum variable detection 68. The controller 22 may also reset the counter 66 after the stagnant period 64 during which the variables 200 detected by the sensor 20 are not outside the predetermined variable parameter 26.

Referring still to FIGS. 7-9, in some aspects, the tensioning device 36 activates the tensioning elements 34 as part of the stabilization protocol 110 in response to both a first variable 200a detected by the first sensor 20a and a second variable 200b detected by the second sensor 20b as being outside the predetermined variable parameter 26. In determining whether to execute the stabilization protocol 110, the controller 22 may compare the first variable 200a with the second variable 200b to determine whether the detected variables 200a, 220b are similarly irregular or whether one of the detected variables 200a, 200b is greater than the other. The controller 22 may subsequently activate the tensioning device 36 to bias the first tensioning element 34a and the second tensioning element 34b when both the first variable 200a and the second variable 200b exceed the predetermined variable threshold 26. In some examples, the location of the respective sensor 20 may influence the detected variable 200. Thus, the controller 22 may individually evaluate the detected variable 200 against the range of intended variables 204, in addition to the predetermined variable parameter 26, to determine whether to execute the stabilization protocol 110.

The controller 22 may activate the stabilization protocol 110 to selectively dampen one or more of the detected variables 200. Additionally or alternatively, the controller 22 may execute the stabilization protocol 110 to dampen a single detected variable 200 when the variable 200 is outside the predetermined variable parameter 26. The stabilization assembly 10 advantageously provides selective dampening effects and, with the controller 22, differentiates between detected variables 200 via comparison with the baseline movement threshold 24 and the predetermined variable parameter 26. In addition, the selective dampening is further refined by the predetermined timeframe 62, which assists the controller 22 in differentiating an outlier variable as compared with a detected variable 200 that is consistently outside the predetermined variable parameter 26 within the predetermined timeframe 62.

With further reference to FIGS. 7-9, the controller 22 is configured to activate the stabilization protocol 110 and, thus, the tensioning device 36 when the detected variable 200 is determined as being outside the predetermined variable parameter 26 and, in some examples, when the detected variable 200 is outside the predetermined variable parameter 26 for the predetermined timeframe 62. In operation, the tensioning device 36 biases the tensioning elements 34 fore-and- aft to translate the mass 38 positioned therebetween. The mass 38 oscillates at the selected attenuation variable 202. The attenuation variable 202 is selected to counter-act or attenuate the detected variable 200 when the detected variable 200 is outside the predetermined variable parameter 26.

In some aspects, the stabilization assembly 10 is configured to respond to variables detected in multiple directions. The mass 38 may thus be oscillated at multiple attenuation variables 202 to neutralize or dampen the detected variable 200 from the sensor 20, when the detected variable 200 is outside the predetermined variable parameter 26. In order to execute the stabilization protocol 110, the tensioning device 36 receives power from the power unit 46 to subsequently bias the tensioning elements 34. The biasing of the tensioning elements 34 and, as a result, the mass 38 is defined by the attenuation variable 202. As noted above, the attenuation variable 200 is configured to neutralize the detected variable 200 when the stabilization protocol 110 is executed to neutralize the unintended movement.

Referring again to FIGS. 1-9, in some examples, the stabilization system 100, mentioned above, may be considered a wearable stabilization system 100 with the first stabilization assembly or device 10a and the second stabilization assembly or device 10b. The first stabilization device 10a includes a first sensor 20a, a first tensioning assembly 30a, a first mass 38a, and a first controller 22a. The second stabilization device 10b includes a second sensor 20b, a second tensioning assembly 30b, a second mass 38b, and a second controller 22b. It is contemplated that the second controller 22b may be communicatively coupled to the first controller 22a, such that the second controller 22b may receive a signal from the first controller 22a corresponding to a variable 200 detected by the first sensor 20a. The second controller 22b may, in response to the detected variable 200, activate a power unit 46b of the second stabilization device 10b to bias the second tensioning assembly 30b.

As with the stabilization assembly 10 described herein, the first controller 22a and the second controller 22b of the respective stabilization devices 10a, 10b are configured to reduce the detected variables 200 by activating the first tensioning assembly 30a and the second tensioning assembly 30b, respectively. In some aspects, the second controller 22b may activate the second tensioning assembly 30b in response to a signal received from the first controller 22a. The stabilization system 100 may further include a third stabilization assembly or device 10c that includes a fluid-filled mass 38c with an adjustment valve and a third controller 22c communicatively coupled with each of the first controller 22a and the second controller 22b. The third controller 22c may be configured to adjust a volume of the fluid-filled mass 38c via the adjustment valve in response to a signal received from at least one of the first controller 22a and the second controller 22b.

With further reference to FIGS. 1-9, the stabilization assembly 10 may be utilized in various settings to assist a wearer in controlling or otherwise neutralizing unintended movements. In some examples, the controller 22 of the stabilization assembly 10 may be communicatively coupled with a third party device 300 via a network 302. The third party device 300 may include, but is not limited to, a vehicle, a user device, and/or a third party receiver. The third party device 300 may be equipped with an electronic control unit 304 that may receive signals from the controller 22 of the stabilization assembly 10 corresponding to execution of the stabilization protocol 110. For example, the controller 22 may notify the electronic control unit 304 that the detected variable 200 is outside the predetermined variable parameter 26 for the predetermined timeframe 62 and, thus, that the stabilization protocol 110 has been executed in response.

The electronic control unit 304 may store the signal received from the controller 22 and monitor for updates from the stabilization assembly 10. Additionally or alternatively, the electronic control unit 304 may communicate with a communication server 306 via the network 302 a notification to monitor the wearer via the third party device 300. The controller 22 may subsequently communicate with the electronic control unit 304 once the detected variable 200 is within the predetermined variable parameter 26, such that any unintended movements have been neutralized and the stabilization protocol 110 has been terminated. In response, the electronic control unit 304 may update the communication server 306 of the status change. The communication server 306 may selectively continue to monitor the wearer through communication with the electronic control unit 304. The wearer may also directly communicate with the communication server 306 via user interfaces integrated with the vehicle 300 through the electronic control unit 304.

Referring still to FIGS. 1-9, in operation, the sensor 20 detects, at 500, a vibration variable 200 and communicates the vibration variable 200 with the controller 22. The controller 22, at 502, determines whether the variable 200 is outside the predetermined variable parameter 26. If the variable 200 is not outside the predetermined variable parameter 26, then the controller 22, at 504, may continue monitoring the detected variables 200. If the variable 200 is outside the predetermined variable parameter 26, then the controller 22, at 506, executes the stabilization protocol 110. In turn, at 508, the tensioning assembly 30 is activated to oscillate or otherwise translate the mass 38.

The controller 22, at 510, continues to monitor the detected variable 200 to determine whether the variable 200 has dropped within the predetermined variable parameter 26. The controller 22 may continue to execute the stabilization protocol 110 until the variable 200 drops within the predetermined variable parameter 26. Once the variable 200 is within the predetermined variable parameter 26, the controller 22, at 512, tapers off the stabilization protocol 110. The controller 22 continuously monitors the detected variables 200 from the sensor 20 to determine whether to execute the stabilization protocol 110.

Referring still to FIGS. 1-9, the stabilization assembly 10 advantageously assists in neutralizing unintended movements by the wearer. Thus, the stabilization assembly 10 may facilitate an improved quality of life by facilitating dampened variables that may otherwise alter day-to-day functionalities. The optionality of the sensor 20 placement further facilitates flexibility in utilizing the stabilization assembly 10. For example, by providing the ability to configure the stabilization assembly 10 with one or both of electrical sensors 20 and mechanical sensors 20, the stabilization assembly 10 may be utilized to detect electrical variables corresponding to brain activity and/or may detect vibration variables. In either configuration, the controller 22 is configured to determine whether the detected variable 200 or variables 200 are outside the predetermined variable parameter 26 to determine whether to execute the stabilization protocol 110.

A number of implementations have been described. Nevertheless, it will be understood that various modifications may be made without departing from the spirit and scope of the disclosure. Accordingly, other implementations are within the scope of the following claims.

The foregoing description has been provided for purposes of illustration and description. It is not intended to be exhaustive or to limit the disclosure. Individual elements or features of a particular configuration are generally not limited to that particular configuration, but, where applicable, are interchangeable and can be used in a selected configuration, even if not specifically shown or described. The same may also be varied in many ways. Such variations are not to be regarded as a departure from the disclosure, and all such modifications are intended to be included within the scope of the disclosure.

What is claimed is:

1. A wearable stabilization assembly comprising:
a housing including a first sensor disposed at a first end of the housing;
a tensioning assembly disposed within the housing and operably coupled to the first sensor via a connection extending through the tensioning assembly and including a first tensioning element, a second tensioning element, and a tensioning device operably coupled to each of the first tensioning element and the second tensioning element;
a mass operably coupled to each of the first tensioning element and the second tensioning element; and
a controller communicatively coupled with the first sensor and, in response to the first sensor detecting a variable outside a predetermined variable parameter, configured to translate the mass via the tensioning assembly.

2. The wearable stabilization assembly of claim 1, wherein the housing includes a first retention feature that includes the first sensor and a second retention feature that includes a second sensor.

3. The wearable stabilization assembly of claim 2, wherein the controller is configured to receive a first vibration variable from the first sensor and a second vibration variable from the second sensor and is configured to compare the first vibration variable with the second vibration variable.

4. The wearable stabilization assembly of claim 3, wherein the controller is configured to activate the tensioning device to bias the first tensioning element and the second tensioning element when both the first vibration variable and the second vibration variable are outside the predetermined variable parameter.

5. The wearable stabilization assembly of claim 1, further including a power unit electrically coupled to each of the first sensor, the controller, and the tensioning device.

6. The wearable stabilization assembly of claim 5, wherein the controller, in response to the predetermined variable parameter detected by the first sensor, is configured to activate the power unit and bias the first tensioning element and the second tensioning element via electrical communication between the power unit and the tensioning device.

7. The wearable stabilization assembly of claim 1, wherein the second tensioning element is disposed on an opposite side of the mass than the first tensioning element, each of the first tensioning element, the mass, and the second tensioning element being operably coupled in series.

8. A wearable stabilization assembly comprising:
a sensor;
a tensioning assembly coupled to the sensor;
a mass operably coupled to the tensioning assembly;
a power unit electrically coupled to the tensioning assembly; and
a controller communicatively coupled with the sensor and configured to activate the power unit to oscillate the mass via the tensioning assembly when a variable detected by the sensor is outside a predetermined variable parameter.

9. The wearable stabilization assembly of claim 8, wherein each of the sensor, the tensioning assembly, the mass, the power unit, and the controller are operably coupled in series.

10. The wearable stabilization assembly of claim 8, wherein the tensioning assembly includes a tensioning device and a first tensioning element operably coupled to the tensioning device, the controller configured to bias the first tensioning element in response to the detected variable being outside the predetermined variable parameter.

11. The wearable stabilization assembly of claim 10, wherein the controller is configured to activate the tensioning device to dampen the detected variable to a variable that is within the predetermined variable parameter.

12. The wearable stabilization assembly of claim 11, wherein the tensioning assembly includes a second tensioning element and the controller is configured to activate the tensioning device to oscillate the mass between the first tensioning element and the second tensioning element to dampen the detected variable.

13. The wearable stabilization assembly of claim 12, wherein the first tensioning element and the second tensioning element are arranged in parallel with the mass.

14. The wearable stabilization assembly of claim 8, wherein the mass is a fluid-filled mass.

15. A wearable stabilization system comprising:
a first stabilization device including a first sensor, a first tensioning assembly, a first mass, and a first controller communicatively coupled to the first sensor and configured to oscillate the first mass via the first tensioning assembly in response to a variable detected by the first sensor being outside a predetermined variable parameter; and
a second stabilization device including a second sensor, a second tensioning assembly, a second mass, and a second controller communicatively coupled to the second sensor and the first controller and configured to oscillate the second mass via the second tensioning assembly in response to at least one of the first controller oscillating the first mass and a variable detected by the second sensor being outside the predetermined variable parameter.

16. The wearable stabilization system of claim 15, wherein the second controller is communicatively coupled to the first controller and configured to receive a signal from the first controller corresponding to the variable detected by the first sensor and is configured to activate a power unit of the second stabilization device to bias the second tensioning assembly.

17. The wearable stabilization system of claim 16, wherein each of the first controller and the second controller is configured to reduce the detected variables by activating the first tensioning assembly and the second tensioning assembly, respectively.

18. The wearable stabilization system of claim 17, wherein the second controller is configured to activate the second tensioning assembly in response to the signal received from the first controller.

19. The wearable stabilization system of claim 15, further including a third stabilization device including a fluid-filled mass, an adjustment valve, and a third controller communicatively coupled with each of the first controller and the second controller, the third controller configured to adjust a volume of fluid within the fluid-filled mass via the adjustment valve in response to a signal received from at least one of the first controller and the second controller.

20. A vehicle comprising an electronic control unit communicatively coupled with the wearable stabilization system of claim 15.
